(19) United States
(12) Patent Application Publication
(10) Pub. No.: US 2004/0072248 A1
(43) Pub. Date: Apr. 15, 2004

(54) Detection of pregnancy

(76) Inventors: Keith Henderson, Upper Hutt (NZ); Alan Rogerson, Mt. Albert (NZ)

Correspondence Address: Samuels, Gauthier & Stevens LLP, Suite 3300, 225 Franklin Street, Boston, MA, 02110, US

(21) Appl. No.: 10/409,973

(22) Filed: Apr. 9, 2003

Related U.S. Application Data
(63) Continuation of application No. PCT/NZ00/00216, filed on Nov. 2, 2000.

(30) Foreign Application Priority Data
Oct. 9, 2000 (NZ) 507422

(51) Int. Cl. G01N 33/53

(52) U.S. Cl. 435/7.1

(57) ABSTRACT

An animal pregnancy test which includes detecting a hormone level in a bodily extract. The bodily extract is tested for a threshold level of the hormone, specifically oestrogen. The test includes a test substance and a material capable of facilitating movement of bodily extract within it. The test substance includes a substance capable of reacting with oestrone sulphate and an indicator dye which is attached to the surface of the substance capable of reacting with oestrone sulphate. The threshold level of the test substance is calibrated to suit specific species and to indicate the desired litter size of that species so that if the detected level of oesterone sulphate exceeds a predetermined threshold level for that species, then there is a high probability of the actual litter size exceeding the calibrated litter size. The apparatus for detecting pregnancy in animals consists of a testing substance, a control test, a sample extractor, and a sample container. The control test consists of a control testing substance which can be either another murine monoclonal antibody or a reagent to any general blood constituent, and an indicator dye.

### FIELD OF THE INVENTION

This invention relates to a method for detecting pregnancy in animals, more particularly a method for detecting pregnancy in animals such as horses, pigs and the like.

### BACKGROUND

Traditionally, pregnancy detection in livestock has utilised a variety of techniques including palpation of the uterus per rectum, ultrasonography and measurement of hormones associated with pregnancy. While reliable, these methods require either specialised veterinary expertise, or sophisticated laboratory facilities. Consequently pregnancy testing by these methods can be costly to livestock owners.

Moreover, measurement of hormones associated with pregnancy usually requires a blood sample to be submitted to a testing laboratory, where the serum needs to be separated from the blood before the test can be performed. It may take several days for a testing laboratory to report the result.

A simple, rapid and cost-effective method of determining pregnancy status which can be performed animal-side would be of considerable benefit to both veterinarians and owners of livestock.

In addition to the determination of pregnancy status, there is also a need for a simple method of determining litter size. The size of a litter can be commercially important to farmers who would benefit greatly from knowing whether the size of the litter was to exceed a certain number. To date, the only method of determining litter size is by the use of ultrasonography or rectal palpation which can only be performed by persons trained in these methods, such as a veterinarian. Determining litter size can again be a costly exercise.

There is a need for a quick and simple method of determining pregnancy status including determining the litter size in livestock, that is accurate, reliable, easy to use and cost-effective.

It is preferable that such a test utilises a biological fluid such as blood, serum, plasma, saliva, urine or milk, can be performed animal-side and be applicable to a wide variety of animals.

### OBJECT

It is an object of the present invention to provide an improved method for diagnosing pregnancy and determining litter size in livestock or a method which will at least provide the public with a useful choice.

### STATEMENT OF INVENTION

In one aspect the invention provides a method of diagnosing pregnancy in animals, which detects levels of hormone.

Preferably said hormone is present in the non-pregnant female but is present at increased levels during pregnancy.

Preferably the hormone is an oestrogen.

More preferably the oestrogen is oestrone sulphate.

Preferably the method for detecting pregnancy is a test.

Preferably the test comprises the use of a testing substance and a material capable of facilitating movement of selective components of a sample to the testing substance.

Preferably the testing substance comprising a substance capable of reacting with oestrone sulphate, a protein carrier conjugated to an oestrogen, and an indicator dye.

Preferably the substance capable of reacting with oestrone sulphate is a murine monoclonal antibody.

Preferably the protein carrier is a protein that can be conjugated to an oestrogen, said oestrogen being capable of recognising the monoclonal antibody to oestrone sulphate. One such example is bovine serum albumin, conjugated to the oestrogen 1,3,5 (10)-estratriene-3-ol-6, 17-dione 6-O-carboxymethyloxime.

Preferably the indicator dye is a substance capable of combining with the monoclonal antibody.

More preferably the indicator dye is such as dyed polystyrene microspheres of preferably mean diameter 0.3 μm.

Preferably the material capable of facilitating movement of oestrone sulphate to the testing substance is a cellulose nitrate membrane supported by polyester, and preferably having a nominal pore size of 3 μm.

Preferably the cellulose nitrate membrane is shaped in a useful form allowing for the movement and detection of a sample, for example a dipstick.

Preferably the test additionally contains a control testing substance capable of reacting with murine monoclonal antibodies, for example an anti-mouse IgG antibody.

In another aspect, the invention provides a kit for detecting pregnancy in animals consisting of a testing substance, a control substance, a material capable of allowing facilitation of bodily extracts within it, a sample extractor and a sample container.

In another aspect the invention provides a method of detecting the litter size of the pregnancy.

Preferably the method of detecting litter size involves setting threshold levels which indicate litter sizes above or below a certain number.

Preferably the method of detecting litter size involves the calibration of raised hormone levels, and comparing the raised hormone levels to the threshold level for a litter size of a particular number.

### Scientific Paper

An abstract on a preferred technique of this invention was disclosed in the proceedings of a conference held by The New Zealand Society of Endocrinology on May 3-5, 2000. A copy of this preliminary report is attached to this specification. The Applicants claim the benefit of the six month grace period from the date of the publication of this abstract.

### BRIEF DESCRIPTION OF THE DRAWINGS

Embodiments of the invention will now be described by way of example only, with reference to the accompanying drawings of:

FIG. 1 Schematic view of testing material used in Example 1.

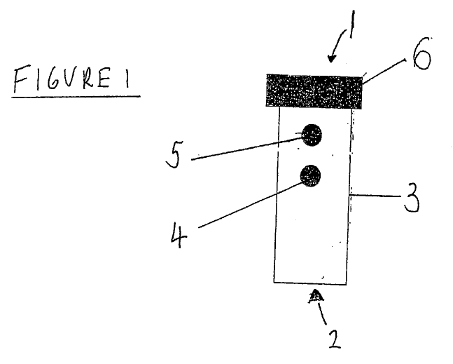

FIG. 2 Graphical Representation of Hormone Levels in Example 1.

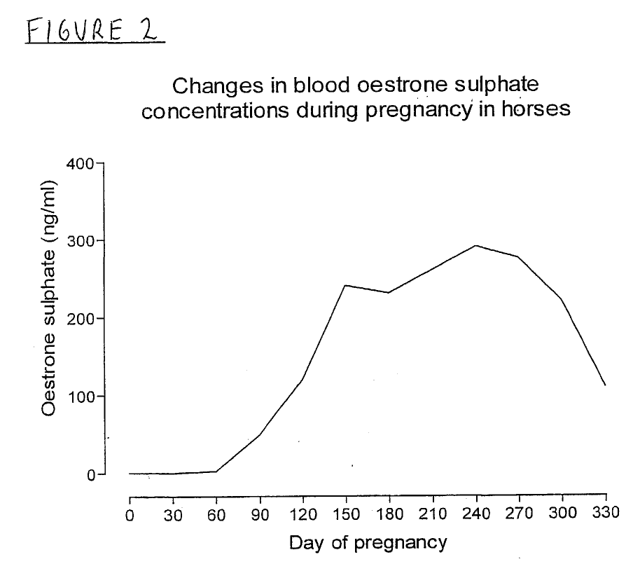

FIG. 3 Graphical Representation of Hormone Levels in Example 2.

### PREFERRED EMBODIMENT

The present invention provides an improved method for detecting the pregnancy status in animals.

The following examples are given by way of illustration only and shall not be taken as being in any way limiting the spirit or scope of the invention.

### EXAMPLE 1

The following description of the invention will be illustrated using the diagnosis of pregnancy in horses as an example. Horses can be tested by this invention from 100 days after mating through to the expected day of foaling.

Referring to the drawings where like numerals designate corresponding parts throughout the several figures.

In this example the test comprises a method for detecting pregnancy in horses consisting of an absorbent strip such as a dipstick, a means to extract a sample, and a sample container.

The dipstick comprises a strip 3 containing a testing substance and an absorbent pad 6 located at the first end 1 of the strip 3.

The strip 3 is preferably a polyester-supported cellulose nitrate membrane having a nominal pore size of preferably 3 μm. A pore size of 3 μm allows for satisfactory movement of selected components of the sample such as the dyed polystyrene microspheres. Those skilled in the art will realise that the strip may be made of different material or have a different nominal pore size and will still be useable in the invention.

A strip 3 having the dimension 45-mm height×5-mm width has been found to be particularly advantageous however other dimensions are also suitable. The dimensions of the strip 3 should be chosen for compatibility with the dimensions of the sample container and the desired duration of the test. Generally, the greater the dimensions the longer the test will take if all other variables remain constant.

On the surface of the strip 3 is the test site 4. The test site 4 consists of a binding substance dotted onto the strip 3 approximately 25 mm from the second end 2. The binding substance contains an oestrogen conjugated to a protein. The oestrogen used should be capable of recognising the monoclonal antibody used to bind oestrone sulphate.

The binding substance used in this example is 1, 3, 5 (10)-estratriene-3-ol-6, 17-dione 6-O-carboxymethyloxime conjugated to the protein bovine serum albumin. Those skilled in the art will realise that a different oestrogen or different protein will still be useable in the invention. The binding substance can be placed on the surface of the strip as a dot or any other pattern, for example a line.

There could be more than one test site 4 located on the strip 3. A series of test sites 4 of different calibrations may be used to indicate varying levels of oestrone sulphate.

The strip 3 would preferably contain a control site 5 in the form of a second dot or mark located on the strip 3. This control site 5 would contain a substance capable of reacting with the murine monoclonal antibody absorbed onto the dyed polystyrene microspheres. One possible substance is anti-mouse IgG antibody. Those skilled in the art will realise that a variety of different substances would be useable in the invention. The control test site 5 will be located in a position sufficiently distant from the test site 4 to avoid confusion.

The control test site 5 will be used to indicate to the user whether he or she has conducted the test correctly. If the test is conducted properly then the control test site 5 should always react positively with the antibody-coated dyed polystyrene microspheres and a coloured zone will appear at the control test site 5.

At the first end 1 of the strip 3 is a pad 6. The pad 6 may preferably be made of 920 μm thick cellulose paper and acts as an absorbent sink. Those skilled in the art will realise that a variety of different media would be useable in the invention as an absorbent sink. The pad 6 helps draw the sample up through the strip 3 past the test site 4 and the control test site 5. The pad 6 can have any dimensions so long as it is capable of fitting onto the first end 1 of the strip 3. The pad 6 should be secured to the first end 1 of the strip 3.

The antibody used in this pregnancy test is preferably a murine monoclonal antibody which recognises the steroid oestrone sulphate. However a polyclonal antibody or a non-murine monoclonal antibody recognising oestrone sulphate could be used as an alternative. In this test the antibody is adsorbed onto blue coloured polystyrene microspheres of mean diameter 0.3 μm. The reason for adsorbing the antibodies onto blue coloured polystyrene microspheres is to give a visual signal when the antibody binds to the binding test site 4 and the control site 5.

Those skilled in the art will realise that the visual signal could be generated by adsorbing the antibody onto alternative carriers, such as different sized or different coloured microspheres, or onto colloidal gold particles. The antibody could also be covalently attached to microspheres or colloidal gold rather than being adsorbed onto the carrier.

The means to extract the sample (hereinafter “the sample extractor”) may be a number of conventional tools capable of puncturing through the skin of a horse and drawing blood. The sample extractor should be capable of accommodating a suitable volume of sample. A suitable volume of sample would be in the range of 0.04 to 1 ml but any sized sample could be used. In addition the sample extractor should be adapted for transfer of the sample to the sample container. An example of a suitable sample extractor is a syringe.

The sample container would be any conventional container capable of housing a sample and a dipstick. The volumetric capacity of the sample container will be commensurate with the size of the sample.

The sample container may optionally have markings on the surface to provide a visual indication of the volume of the sample. There may additionally be a mark that indicates the minimum sample volume required.

As an alternative, the functions of both the sample extractor and sample container can be combined into one tool. This tool would have the dual function of extracting the sample and accommodating the sample while the test is being conducted.

### Preparation of Dipsticks

Dipstick strips of dimensions 45×5 mm are prepared out of sheets of polyester supported cellulose nitrate membranes, pore size 3 μm. 1,3,5 (10)-estratriene-3-ol-6,17-dione 6-O-carboxymethyloxime conjugated to the protein bovine serum albumin (oestrone CMO-BSA) is dissolved in coating buffer (50 mmol/l carbonate-bicarbonate buffer, pH 9.6) at a suitable concentration, for example from 0.1 to 2 mg/ml though other concentrations would be useable. Using a Hamilton syringe or other dispensing device, an aliquot of the conjugated solution is ‘dotted’ onto each strip 25 mm from the bottom edge. After drying for 30 minutes at 37° C., the strips are immersed in a blocking solution (for example coating buffer containing 1% polyvinylpyrrolidone, average molecular weight 10,000, and 0.5% Triton X-100) for 30 minutes at room temperature. The strips are washed with 5 changes of distilled water, and dried at 37° C. for 30 minutes. A 15-mm×5-mm absorbent sink, made from 920 μm thick cellulose paper, is attached with adhesive tape 10 mm from the first end of each strip. These prepared dipsticks are stable for several months when stored at room temperature with a silica gel desiccant.

### Preparation of Antibody-coated Microspheres

Uniform, blue-dyed, polystyrene microspheres of mean diameter 0.31 μm are washed twice in coating buffer to remove surfactant, and re-suspended by sonication in coating buffer at a concentration of 10% (w/v) solids. Protein G affinity chromatography is used to purify from ascites fluid the IgG fraction of a murine monoclonal antibody recognising oestrone sulphate. The purified oestrone sulphate antibody IgG is re-suspended in coating buffer at an appropriate concentration, for example 20 to 200 μg protein/ml and 0.5 ml aliquots added to tubes containing a further 0.4 ml of coating buffer. A 0.1 ml aliquot of the washed, resuspended blue-dyed microspheres is then added to each tube with rapid mixing, and the tubes incubated with gentle mixing overnight at 4° C. to allow the antibody to absorb onto the microspheres. (The amount of antibody incubated with the microspheres is chosen so that the antibody coated microspheres will not interact with the test substance at control site 4 in the presence of oestrone sulphate at a concentration above 20 ng/ml). The microspheres are separated from unbound antibody by centrifugation, and blocked for 30 minutes at room temperature with 1% (w/v) BSA in coating buffer. The microspheres are washed twice with washing buffer (10 mM phosphate buffer containing 0.8% NaCl and 0.05% Tween 20, pH 7.4) and once with storage buffer (100 mM borate buffer, pH 8.2 containing 0.1% BSA, 0.05% Tween 20 and 0.01% thimerosal), before being finally re-suspended with sonication in storage buffer at a concentration of 1% (w/v) solids, and stored at 4° C. The antibody coated microspheres can be used either in solution, or dried directly onto the dipsticks below the test site 4 and control site 5, or dried onto a release pad which is attached to the dipsticks below the test site 4 and control site 5.

Instructions for Use

If the sample is to be blood then the tester must firstly draw a blood sample. The blood sample can be taken from any area of the horse having a good blood supply. Around the nose area or the jugular vein is particularly recommended

The blood sample should be transferred to the sample container; this will not be necessary if the sample extractor and sample container are combined into one tool. If a serum sample is to be tested, the sample should be placed into the sample container. If the antibody-coated microspheres have not been dried onto the dipsticks, either directly or via a release pad, then an aliquot of antibody-coated microspheres should be added to the sample container.

For testing either a blood or serum sample the second end 2 of the strip 3 should be placed into the sample container bringing the sample into contact with the strip 3. The liquid components will move up the dipstick by capillary action, and as the solvent front progresses up the dipstick an immuno-reaction will occur. If the blood/serum sample is from a pregnant mare, then the oestrone sulphate concentration in the sample will be greater than 20 ng/ml. The oestrone sulphate in the sample will bind to the antigen-binding sites on the antibody adsorbed onto the coloured microspheres. This will prevent the antibody from binding to the oestrogen binding substance located at the test site 4. The antibody-coated microspheres will flow past the test site 4 and no colour will be visible at this site. The antibody-coated microspheres will be recognised by the control substance located at control site 5 and a blue colour will form as the coloured microspheres pass over this site.

Pregnancy will thus be detected by a visible blue colour being present at control test site 5, but no colour at test site 4. Conversely, if the blood/serum sample is from a non-pregnant mare the oestrone sulphate concentration in the sample will be less than 10 ng/ml. This concentration of oestrone sulphate will be insufficient to occupy all the antigen-binding sites on the antibody-coated microspheres. As the antibody-coated microspheres pass over the test site 4, some antibody will interact with oestrogen located at this site and a visible blue colour will develop. Some antibody will also react with the control substance at site 5, and a visible blue colour will develop at this site too. A non-pregnant mare will thus be diagnosed by a visible blue colour being formed at the test site 4 and control test site 5.

The strip 3 should be read within 10-15 minutes of placing it in the sample, which will give a more accurate reading. The duration in this example is 10-15 minutes but tests of different durations are possible and depend on factors such as the dipstick material and the length of the dipstick amongst other things.

If the test has been conducted properly then the control test site 5 should always develop a blue colour. If the control test site 5 has not developed colour after 15 minutes then the test should be repeated.

### EXAMPLE 2

The test of the present invention can also be used to detect pregnancy in pigs. The blood oestrone sulphate concentrations in pigs are lower than those in horses but may still be tested for using the test of the present invention. Small variations must be made to account for the lower levels of oestrone sulphate, notably the microspheres will need to be coated with a different concentration of antibody.

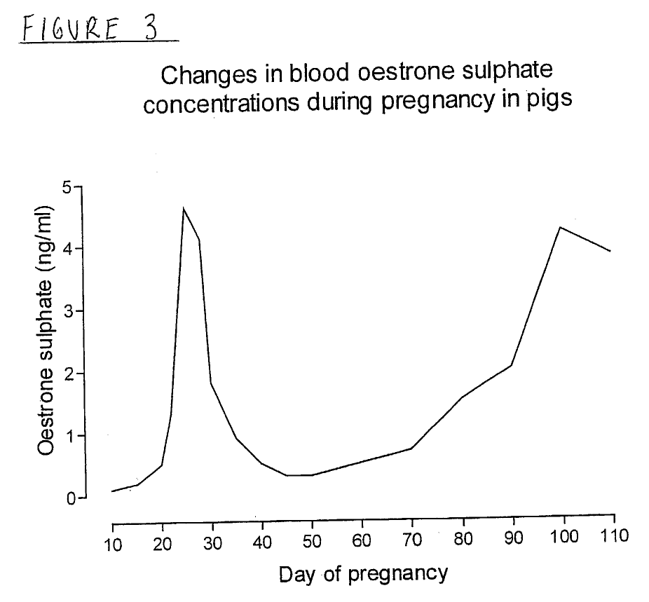

In pregnant pigs there is a transient rise in blood oestrone sulphate concentrations above 1 ng/ml between days 20 and 30 post-breeding. Levels then drop to levels similar to those of non-pregnant pigs, i.e. less than 0.5 ng/ml, before rising again around 80 days after mating (FIG. 3). If levels of oestrone sulphate above 1 ng/ml can be detected in a blood sample collected between 20 and 30 days post-mating a pig can be diagnosed as pregnant. In addition it has been found that the blood concentration of oestrone sulphate found in pregnant pigs between 20 and 30 days post-mating is correlated with litter size. For example, concentrations between 1 and 2.5 ng/ml are generally indicative of a low litter size (<6 conceptuses) while concentrations above 3 ng/ml are generally indicative of a high litter size (>6 conceptuses). When using the invention in pigs, the test will be calibrated such that no visual dot will be appear at test site 4 if the pig is pregnant and likely carrying a high litter size, that is >6 conceptuses. A dot appearing at test site 4 will indicate that the pig is either not pregnant, or pregnant but likely carrying a small litter i.e. <6 conceptuses.

When testing for pregnancy and litter size in pigs, it is important that the test be conducted at the correct time, which is between 20 and 30 days post-mating.

### Advantages

The examples described hereto provide consumers with a quick and easy test for determining pregnancy in animals. Providing results as to the pregnancy status in animals within such a short time frame is of great convenience to consumers.

The test is simple to use and can be completed on whole blood samples. This eliminates the need for the testing to be conducted in laboratories. Because the test can be conducted anywhere, the tester can simply take samples from the stock on site.

In addition to being able to determine pregnancy status in pigs, the test also allows litter size to be estimated. This is of considerable benefit to pig farmers as it allows the cost-effectiveness of maintaining the pregnancy through to term to be calculated.

### Variations

The invention could be used on animals other than those discussed in examples 1 and 2. Other mammalian animals such as sheep, cows, deer, buffalo, goats and camelids could also be tested for pregnancy. The testing procedure may vary for different animals depending on the stage of gestation the oestrone sulphate levels increase, and how much the levels increase by.

The test of the invention can be used in serum samples as well as whole blood samples. Examples 1 and 2 have described the testing of blood samples because of the clear advantage this has to farmers testing their stock on site. Urine and faecal samples also contain oestrone sulphate and could,be tested. Oestrone sulphate is also present in saliva but at low concentrations. A more sensitive test could be used for testing oestrone sulphate at this lower concentration.

There are various materials capable of facilitating movement of selective component of a sample used in place of those described hereto. Example 1 describes a dipstick of certain dimensions made from cellulose nitrate, supported by polyester, and having a nominal pore size of 3 μm. As a variation, the dipstick could be of different dimensions allowing for a test of longer duration time.

It could also be in the form of a disc with marked concentric circles around the centre where the sample is placed. The test site and control site could be located at varying positions around the concentric circles. After the sample is placed in the centre of the disc it will move by capillary action outwards through the testing sites.

There could be a different number of test sites and control test sites used. There could be two or more of each of these sites located on the strip.

Throughout the description and claims of this specification the word “comprise” and variations of that word, such as “comprises” and “comprising”, are not intended to exclude other additives, components, integers or steps.

We claim:

1. A method of determining pregnancy status in an animal by detecting a hormone in a bodily extract, wherein the bodily extract is tested for a threshold level of the hormone, and the hormone tested for is an oestrogen.

2. A method of determining pregnancy status as claimed in claim 1, wherein the oestrogen tested for is oestrone sulphate.

3. A method of determining pregnancy status as claimed in claim 1 characterised by a test comprising a test substance and a material capable of facilitating movement of bodily extract within it, wherein the test substance includes (a) a substance capable of reacting with oestrone sulphate and (b) an indicator dye which is attached to the surface of the substance capable of reacting with oestrone sulphate.

4. A method of determining pregnancy status as claimed in claim 1 wherein the threshold level of the test substance is calibrated to suit specific species and to indicate the desired litter size of that species so that if the detected level of oestrone sulphate exceeds a predetermined threshold level for that species, then there is a high probability of the actual litter size exceeding the calibrated litter size.

5. A method of determining pregnancy status as claimed in claim 1, wherein, the bodily extract is whole blood and the time taken for the test to be conducted is a period not more than 30 minutes.

6. A method of determining pregnancy status as claimed in claim 3 wherein the material capable of facilitating movement of a bodily extract within it contains or consists of cellulose nitrate shaped in a form allowing for movement and detection of oestrone sulphate in a sample, such as a dipstick.

7. A method of determining pregnancy status as claimed in claim 1 wherein the substance capable of reacting with oestrone sulphate is for example a murine monoclonal antibody.

8. A method of determining pregnancy status as claimed in claim 1, wherein the murine monoclonal antibody is coupled with a protein-hormone conjugate, bovine serum albumin conjugated to 1,3,5 (10)-estratriene-3-ol-6,17-dione 6-0-carboxymethyloxime.

9. A method of detecting pregnancy status as claimed in claim 1, wherein the testing substance additionally contains a control test site, wherein the control test site is located superior to the test site when the dipstick is in use and contains a substance capable of reacting with the murine monoclonal antibody adsorbed onto the coloured polystyrene sphere, such as anti-mouse IgG antibody.

10. Apparatus for detecting pregnancy in animals consisting of a testing substance, a control test, a sample extractor, and a sample container wherein the testing substance is an antibody to an oestrogene connected to the surface of an indicator dye, a protein-hormone conjugate, and the control test consists of a control testing substance which can be either another murine monoclonal antibody or a reagent to any general blood constituent, and an indicator dye.
